(19) United States
(12) Patent Application Publication
(10) Pub. No.: US 2021/0315830 A1
(43) Pub. Date: Oct. 14, 2021

(54) A CURCUMIN LOADED STABILIZED POLYMERIC NANOPARTICLES WITH INCREASED SOLUBILITY AND PHOTO-STABILITY AND A GREEN PROCESS FOR THE SYNTHESIS THEREOF

(71) Applicant: COUNCIL OF SCIENTIFIC AND INDUSTRIAL RESEARCH, New Delhi (IN)

(72) Inventors: Amitabha Acharya, Himachal Pradesh (IN); Avnesh Kumari, Himachal Pradesh (IN); ANIKA Guliani, Himachal Pradesh (IN); Sanjay Kumar, Himachal Pradesh (IN)

(21) Appl. No.: 17/272,062

(22) PCT Filed: Apr. 9, 2019
(86) PCT No.: PCT/IN2019/050288

§ 371 Date: Feb. 26, 2021

(30) Foreign Application Priority Data
Aug. 31, 2018 (IN) 201811032671

(51) Int. Cl. A61K 9/51; A61K 31/12

(52) U.S. Cl. CPC A61K 9/5176; A61K 9/5153; B82Y 5/00; A61K 31/12; A61K 9/5192

(57) ABSTRACT

The present invention explored PLGA NPs synthesis using plant extracts as surfactants by nanoprecipitation process. PLGA NPs synthesized using Camellia sinensis, Dendrocalamus hamiltonii, Ficus palmata and Rubus ellipticus leaf extracts were further explored for encapsulation, controlled and sustained release of well-known antioxidant molecule, curcumin. This plant extract based nanoprecipitation process for the synthesis of PLGA NPs can be further explored for encapsulation of many other antioxidant molecules of therapeutic importance.

### FIELD OF THE INVENTION

The present invention relates in general to the field of green process based on plant leaf extract based synthesis of curcumin loaded poly (D,L-lactide-co-glycolide) nanoparticles (PLGA NPs) for controlled and sustained release of the same. In particular, the present invention relates to the synthesis and loading of curcumin on poly (D,L-lactide-co-glycolide) nanoparticles, synthesized using leaf extracts of Camellia sinensis, Dendrocalamus hamiltonii, Ficus palmata and Rubus ellipticus.

### BACKGROUND OF THE INVENTION

Poly (D,L-lactide-co-glycolide) nanoparticles (PLGA) have been synthesized using synthetic stabilizers. Synthetic stabilizers raise concerns about the toxicity and health issues. Poly (D,L-lactide-co-glycolide) nanoparticles have been synthesized by solvent evaporation and nanoprecipitation method. These methods use synthetic stabilisers which confer toxicity to the poly (D,L-lactide-co-glycolide) nanoparticles. Prior arts have used synthetic stabilisers like polyvinyl alcohol, polyvinylpyrrolidone etc. for the synthesis of Poly (D,L-lactide-co-glycolide) nanoparticles.

Reference is made to WO 2016/167732 which describes curcumin and piperine loaded biopolymer based nanodelivery systems.

US patent No. US2014/0065061A1 have disclosed liposomal Poly (D,L-lactide-co-glycolide) sustained release nanocurcumin formulation for minimising QT (delayed ventricular repolarisation) prolongation in cancer therapy.

US patent No. 2011/0190399 discloses method for producing curcumin nanoparticles.

US patent No. 2010/0290982A1 reported method for preparing curcumin loaded nanoparticles poly (D,L-lactide-co-glycolide) nanoparticles.

US patent No. 2006/0067998A1 discloses liposomal curcumin for cancer treatment. CN1736369 describes a curcumin oil emulsion and injection for improving bioavailability of curcumin.

U.S. Pat. No. 9,023,395B2 discloses curcumin loaded poly (D,L-lactide-co-glycolide) nanoparticles for targeted cancer nanotherapeutics. It discloses the compositions and methods of making an activated polymeric nanoparticle for targeted drug delivery that includes a biocompatible polymer and an amphiphilic stabilizing agent non-covalently associated with a spacer compound that includes at least one electrophile that selectively reacts with any nucleophilic on a targeting agent and places the targeting agent on the exterior surface of a biodegradable nanoshell, wherein an active agent is loaded with the nanoshell. The amphiphilic stabilizing agent is a polyol.

Pillai et al., 2015 reported curcumin entrapped poly (D,L-lactide-co-glycolide)-polyethyleneglycol nanoparticles showed enhanced anticancer activity. Kasinathan et al., 2015 have reported polycaprolactone-based in situ implant containing curcumin-poly (D,L-lactide-co-glycolide) nanoparticle.

Verderio et al., 2013 have reported intracellular drug release from curcumin loaded poly (D,L-lactide-co-glycolide) nanoparticles induced G2/M block in breast cancer cells.

Mathew et al., 2012 have reported curcumin loaded PLGA nanoparticles for use in Alzheimer's disease.

Punfa et al., 2012 have reported enhanced cellular uptake of curcumin loaded PLGA nanoparticles by conjugation with anti-P-glycoprotein.

Xie et al., 2011 reported poly (D,L-lactide-co-glycolide) nanoparticles for improving the bioavailability of curcumin.

Alshashan, A., 2014 have reported encapsulation of cucurbitacin in PLGA nanoparticles synthesized using PVA as stabiliser.

Narayanan et al., 2010 have encapsulated grape seed extract in poly (D,L-lactide-co-glycolide) nanoparticles.

Stecanella et al., 2012 have reported poly (D,L-lactide-co-glycolide) nanoparticles synthesized using poloxamer.

Nam et al., 2012 have reported poly (D,L-lactide-co-glycolide) nanoparticles synthesized using polyacrylic acid.

Kumari et al., 2012 have documented poly(D,L-lactide) nanoparticles synthesized using plant extracts.

Kesente et al., 2017 have reported encapsulation of olive leaves extracts in polylactide nanoparticles.

Prior arts have used synthetic stabilizers like polyvinyl alcohol, polyvinylpyrrolidone, poloxamer and polyacrylic acid for the synthesis of poly (D,L-lactide-co-glycolide) nanoparticles. Prior arts also have used poly (D,L-lactide-co-glycolide) nanoparticles for encapsulating plant extracts. Prior arts have failed to document use of plant extracts as stabiliser for the synthesis of poly (D,L-lactide-co-glycolide) nanoparticles. Prior arts have failed to provide alternate plant based stabilizers which are safe and are cost effective in synthesizing poly (D,L-lactide-co-glycolide) nanoparticles in the size range 68 to 206 nm. In recent years, stabilizers from bio resources have gathered significant attention of scientific community. This has prompted to switch over to green synthesis of poly (D,L-lactide-co-glycolide) nanoparticles using leaf extracts of plants. Plant leaf extracts contain secondary metabolites of therapeutic importance and have been proved as good stabilisers for metallic and polymeric nanoparticles. The ability of leaf extracts of four medicinally important plants viz., Camellia sinensis, Ficus palmata, Rubus ellipticus and Dendrocalamus hamiltonii have been screened. Cammelia sinensis leaf extracts contain flavanols, hydroxyl-4-flavanols, anthocyanins, flavones and phenolic acids. Important and characteristic tea polyphenols are the flavanols of which catechins (flavan-3-ols) are pre-dominant and the major ones are epicatechin, epicatechingallate, epigallocatechin, epigallocatechin gallate, catechin, and gallocatechin. Tea leaves have antioxidant, antimicrobial and anticancer properties. It also helps in reducing the risk of Alzheimer's and Parkinson's disease. It also plays vital role in lowering the risk of type-II diabetes. Consumption of green tea leaves also reduces the risk of cardiovascular diseases.

Dendrocalamus hamiltonii leaves extracts are rich in flavones, lactones, and phenolic acids, and the representative compounds includes orientin, homoorientin, vitexin, isovitexin, hydroxyl-coumarin, chlorogenic acid, caffeic acid, and ferulic acid. Consumption of Dendrocalamus hamiltonii leaves tea helps in reducing sugar level and also helps to set digestive system. Dendrocalamus hamiltonii leaves also have the property of wound healing, antioxidant and lowering blood pressure. Rubus ellipticus leaf extracts contain tannins, kaempferol, phenolic acids, triterpenes, ellagic acid, quercetin and kaempferol. Leaf extracts of Rubus ellipticus have many biological activities such as antioxidant, anti-inflammatory, analgesic, and antipyretic activities. Aqueous extracts of leaves of Ficus palmata contain mainly flavonoids. Many Ficus species are used in folk medicine as antioxidant, anti-tumor, anti-inflammatory and tonic medicament.

Curcumin has been shown to exhibit antioxidant, anti-inflammation, anti-diabetic, anti-carcinogenic, and anti-angiogenesis activities. But therapeutic use of curcumin is limited due to poor bioavailability, poor pharmacokinetics, poor aqueous solubility, degradability at neutral to basic pH conditions. Poly (D,L-lactide-co-glycolide) nanoparticles synthesized using leaf extracts of Camellia sinensis, Dendrocalamus hamiltonii, Ficus palmata, and Rubus ellipticus have the potential for enhancing the solubility and activity of curcumin.

In the present invention plant leaf extracts of Camellia sinensis, Dendrocalamus hamiltonii, Ficus palmata, and Rubus ellipticus have been used as stabilisers for the synthesis of poly (D,L-lactide-co-glycolide) nanoparticles by nanoprecipitation method.

### Abbreviations

Curcumin: CURCU,

Curcumin loaded poly (D,L-lactide-co-glycolide) nanoparticles—CURCU-PLGA NPs

Poly (D,L-lactide-co-glycolide) nanoparticles—PLGA NPs

Polyethyleneglycol—PEG

Cammelia sinensis leaf extracts—CSE

Dendrocalamus hamiltonii leaves extracts—BE

Rubus ellipticus leaf extracts—RE

Ficus palmata leaves extracts—FE

Curcumin-PLGA nanoparticles using CSEs—CSE-CURCU-PLGA NPs

Curcumin-PLGA nanoparticles using BEs—BE-CURCU-PLGA NPs

Curcumin-PLGA nanoparticles using REs—RE-CURCU-PLGA NPs

Curcumin-PLGA nanoparticles using FEs—FE-CURCU-PLGA NPs

High performance liquid chromatography—HPLC

Ultraviolet Visible spectroscopy—UV-Vis

Transmission electron microscopy—TEM

Dynamic light scattering—DLS

### Objectives of the Invention

It is therefore an object of the present invention to use leaf extracts of Camellia sinensis, Dendrocalamus hamiltonii, Ficus palmata and Rubus ellipticus for synthesis of poly (D,L-lactide-co-glycolide) nanoparticles.

Another object of the present invention is the loading of curcumin to poly (D,L-lactide-co-glycolide) nanoparticles PLGA NPs (CSE-CURCU-PLGA, BE-CURCU-PLGA, RE-CURCU-PLGA and FE-CURCU-PLGA).

Yet another object of the present invention is the optimization and characterization of curcumin loaded poly (D,L-lactide-co-glycolide) nanoparticles PLGA NPs (CSE-CURCU-PLGA, BE-CURCU-PLGA, RE-CURCU-PLGA and FE-CURCU-PLGA).

Still another object of the present invention is optimization of curcumin loaded poly (D,L-lactide-co-glycolide) nanoparticles for controlled and sustained release of the same.

### SUMMARY OF THE INVENTION

Accordingly, the present invention provides stabilized polymeric nanoparticles, synthesized using leaf extracts as stabilizers, having particle size in the range of 68-206 nm, wherein the ratio of polymer to leaf extracts is in the range of 5:1 (w/v).

In a preferred embodiment of the present invention, the leaf extract is obtained from plants selected from the group consisting of Camellia sinensis, Dendrocalamus hamiltonii, Ficus palmata and Rubus ellipticus.

In another preferred embodiment of the present invention, the polymer is poly (D,L-lactide-co-glycolide) (PLGA).

In yet another embodiment of the present invention, the polymeric nanoparticles are loaded with 7.8-15.6% curcumin.

In still another embodiment of the present invention, the curcumin loaded polymeric nanoparticles have increased solubility of 13±5 folds and photo-stability of 22±7.5%.

In yet another embodiment of the present invention, the curcumin loaded polymeric nanoparticles have slow and sustained release of curcumin of up to 45% after 4 h.

The present invention provides a process for the synthesis of stabilized PLGA nanoparticles comprising:
a) dissolving freshly crushed leaves in double distilled water (DDW) and boiling up to 30 minutes;
b) cooling the solution to room temperature (25-30° C.) and filtering to obtain the supernatant;
c) dissolving PLGA in acetone under stirring for 2 hours;
d) adding the supernatant obtained in step b to PLGA solution of step c and stirring between 40-50° C. for 6-10 hours, wherein PLGA:plant extract is at a ratio of 5:1 w/v and
e) removing acetone and centrifuging at about 13500 rpm at 10° C. for 20 minutes for collecting stabilized PLGA nanoparticles.

In a preferred embodiment of the present invention, the leaves are obtained from plants selected from the group consisting of Camellia sinensis, Dendrocalamus hamiltonii, Ficus palmata and Rubus ellipticus.

In one embodiment of the present invention, curcumin was loaded in PLGA nanoparticles by dissolving curcumin along with PLGA in acetone at a ratio of PLGA:curcumin at 100:5.

The present invention provides a curcumin loaded stabilized PLGA nanoparticles synthesized by the above said process.

The present invention provides a novel process for the synthesis of curcumin loaded Camellia sinensis, Dendrocalamus hamiltonii, Ficus palmata, and Rubus ellipticus leaf extracts stabilized poly (D,L-lactide-co-glycolide) nanoparticles for slow and sustained release of the same.

The present invention provides for optimization and characterization of curcumin loaded poly (D,L-lactide-co-glycolide) nanoparticles using nanoprecipitation method while determining formulation variables like poly (D,L-lactide-co-glycolide) content, Camellia sinensis, Dendrocalamus hamiltonii, Ficus palmata, and Rubus ellipticus leaf extracts concentration and their influence on physiochemical properties of poly (D,L-lactide-co-glycolide) nanoparticles.

In one embodiment the present invention provides a process for the preparation of leaf extracts of Camellia sinensis, Dendrocalamus hamiltonii, Ficus palmata, and Rubus ellipticus.

In another embodiment the present invention provides a nanoprecipitation process using leaf extracts of Camellia sinensis, Dendrocalamus hamiltonii, Ficus palmata, and Rubus ellipticus for the synthesis of blank poly (D,L-lactide-co-glycolide) nanoparticles.

In a preferred embodiment the present invention provides a nanoprecipitation process using leaf extracts of Camellia sinensis for the synthesis of curcumin loaded poly (D,L-lactide-co-glycolide) nanoparticles.

In another preferred embodiment the present invention provides a nanoprecipitation process using leaf extracts of Dendrocalamus hamiltonii for the synthesis of curcumin loaded poly (D,L-lactide-co-glycolide) nanoparticles.

In a preferred embodiment the present invention provides a nanoprecipitation process using leaf extracts of Rubus ellipticus for the synthesis of curcumin loaded poly (D,L-lactide-co-glycolide) nanoparticles.

In another preferred embodiment the present invention provides a nanoprecipitation process using leaf extracts of Ficus palmata for the synthesis of curcumin loaded poly (D,L-lactide-co-glycolide) nanoparticles.

In still another embodiment of the present invention, 8 formulations of poly (D,L-lactide-co-glycolide) nanoparticles have been characterized by TEM and DLS and are listed in Table 1.

In yet another embodiment of the present invention, encapsulation efficiency of four curcumin loaded of poly (D,L-lactide-co-glycolide) nanoparticles has been determined using HPLC and are listed in Table 1.

In still another embodiment of the present invention, four formulations listed in Table 1 have been used for slow and sustained release of curcumin. In vitro release profile of curcumin has been studied using high performance liquid chromatography.

In yet another embodiment of the present invention, curcumin loaded Camellia sinensis leaf extracts stabilized poly (D,L-lactide-co-glycolide) nanoparticles showed slow and sustained release of curcumin from nanoparticles.

In still another embodiment of the present invention, curcumin loaded Camellia sinensis leaf extracts stabilised poly (D,L-lactide-co-glycolide) nanoparticles showed 22±7.5% increase in photostability as compared to pure curcumin.

In yet another embodiment of the present invention, curcumin loaded Camellia sinensis leaf extracts stabilised poly (D,L-lactide-co-glycolide) nanoparticles showed 13±5 fold increase in solubility as compared to pure curcumin.

### BRIEF DESCRIPTION OF THE DRAWINGS

FIG. 1. CSE synthesized PLGA NPs (a) UV-Vis spectra of CSE-PLGA and CSE-CURCU-PLGA NPs, (b) TEM image of CSE-PLGA and CSE-CURCU-PLGA NPs, (c) DLS size and zeta potential of CSE-PLGA NPs and (d) DLS size and zeta potential of CSE-CURCU-PLGA NPs

FIG. 2. BE synthesized PLGA NPs (a) UV-Vis spectra of BE-PLGA and BE-CURCU-PLGA NPs, (b) TEM image of BE-PLGA and BE-CURCU-PLGA NPs, (c) DLS size and zeta potential of BE-PLGA NPs and (d) DLS size and zeta potential of BE-CURCU-PLGA NPs.

FIG. 3. RE synthesized PLGA NPs (a) UV-Vis spectra of RE-PLGA and RE-CURCU-PLGA NPs, (b) TEM image of RE-PLGA and RE-CURCU-PLGA NPs, (c) DLS size and zeta potential of RE-PLGA NPs and (d) DLS size and zeta potential of RE-CURCU-PLGA NPs.

FIG. 4. FE synthesized PLGA NPs (a) UV-Vis spectra of FE-PLGA and FE-CURCU-PLGA NPs, (b) TEM image of FE-PLGA and FE-CURCU-PLGA NPs, (c) DLS size and zeta potential of FE-PLGA NPs and (d) DLS size and zeta potential of FE-CURCU-PLGA NPs.

FIG. 5. HPLC calibration curve of pure CURCU.

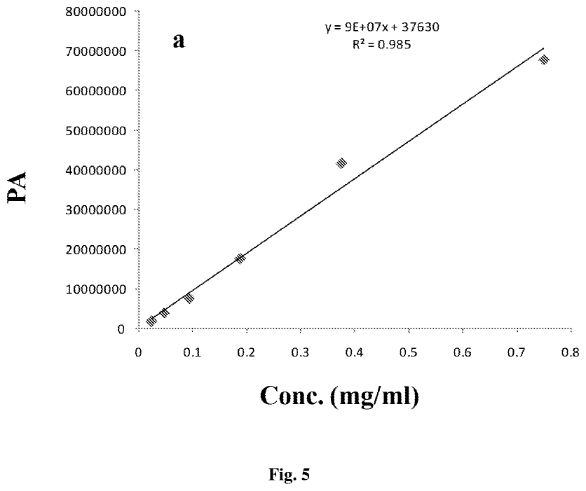

FIG. 6. HPLC results of in vitro % cumulative release profile for CSE-CURCU-PLGA NPs, BE-CURCU-PLGA NPs, RE-CURCU-PLGA NPs and FE-CURCU-PLGA NPs at different time interval

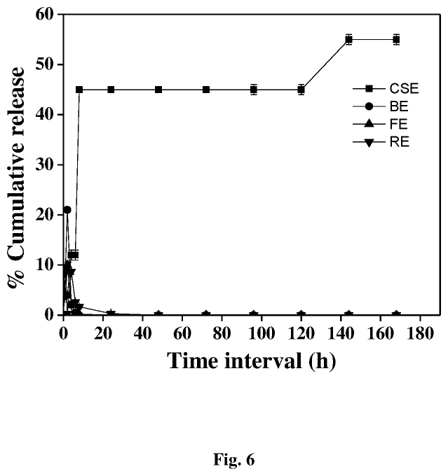

FIG. 7. Graph for % photodegradibility of pure CURCU and CSE-CURCU-PLGA NPs.

FIG. 8. Graph for solubility (mg/ml) of pure CURCU and CSE-CURCU-PLGA NPs in double distilled water

### DETAILED DESCRIPTION OF THE INVENTION

The present invention describes the optimization of nanoprecipitation process for the synthesis of poly (D,L-lactide-co-glycolide) nanoparticles using plant leaf extracts of Camellia sinensis, Dendrocalamus hamiltonii, Rubus ellipticus and Ficus palmata. Leaf extracts of Camellia sinensis, Dendrocalamus hamiltonii, Rubus ellipticus and Ficus palmata were prepared by dissolving 40 g of leaves of each plant (Camellia sinensis, Dendrocalamus hamiltonii, Rubus ellipticus, and Ficus palmata) in 500 ml of distilled water respectively. The solution was boiled for ˜30 minutes. The above solution was cooled to room temperature and filtered through Whatman®-42 filter paper. The supernatant was termed as extract (Camellia sinensis, Dendrocalamus hamiltonii, Rubus ellipticus and Ficus palmata for the respective cases) and used for poly (D,L-lactide-co-glycolide) nanoparticles synthesis.

In accordance to the first objective of the invention, the present invention describes the synthesis of blank poly (D,L-lactide-co-glycolide) nanoparticles using leaf extracts of Camellia sinensis, Dendrocalamus hamiltonii, Rubus ellipticus and Ficus palmata by nanoprecipitation process.

Another aspect of the present invention deals with the method of preparing PLGA NPs using leaf extracts of Camellia sinensis, Dendrocalamus hamiltonii, Rubus ellipticus and Ficus palmata by nanoprecipitation method comprising: (a) formation of a solution involving an organic solvent and poly (D,L-lactide-co-glycolide) polymer and stirring the solution, (b) formation of another solution comprising of leaf extracts, (c) forming a solution by adding second solution to the first solution, (d) stirring the mixture on stirrer for 24 hours and finally (e) centrifugation at 13500 rpm for 20 minutes at 10° C. to recover NPs.

The present invention also describes, method for producing poly (D,L-lactide-co-glycolide) nanoparticles using leaf extracts, and the organic solvent used is 10 ml acetone for all the formulations which was completely removed afterwards.

In yet another aspect of the present invention, method for producing poly (D,L-lactide-co-glycolide) nanoparticles is described where the leaf extracts used in step (b) are 20 ml of leaf extracts selected from Camellia sinensis, Dendrocalamus hamiltonii, Rubus ellipticus and Ficus palmata. Four formulations of poly (D,L-lactide-co-glycolide) nanoparticles have been prepared and the extract concentration used is 8% (w/v).

In still another aspect of the present invention, method for producing blank poly (D,L-lactide-co-glycolide) nanoparticles using leaf extracts of Camellia sinensis, Dendrocalamus hamiltonii, Rubus ellipticus and Ficus palmata is described, the poly (D,L-lactide-co-glycolide) content used in step (a) is 100 mg.

In accordance with the second objective, the present invention described detailed method for the synthesis of four formulations of curcumin loaded poly (D,L-lactide-co-glycolide) nanoparticles using leaf extracts of Camellia sinensis, Dendrocalamus hamiltonii, Rubus ellipticus and Ficus palmata.

Yet another aspect of the present invention deals with the method of preparing curcumin loaded poly (D,L-lactide-co-glycolide) nanoparticles using leaf extracts of Camellia sinensis, Dendrocalamus hamiltonii, Rubus ellipticus and Ficus palmata by nanoprecipitation method comprising: (a) formation of a solution comprising an organic solvent, poly (D,L-lactide-co-glycolide), and curcumin and stirring the solution, (b) formation of another solution comprising of leaf extracts of Camellia sinensis, Dendrocalamus hamiltonii, Rubus ellipticus and Ficus palmata, (c) forming a solution by adding second solution to the first solution, (d) stirring the mixture on stirrer for 24 hours and finally (e) centrifugation at 13500 rpm for 20 minutes at 10° C. to recover nanoparticles.

In still another aspect of the present invention, method for producing curcumin loaded poly (D,L-lactide-co-glycolide) nanoparticles using leaf extracts of Camellia sinensis, Dendrocalamus hamiltonii, Rubus ellipticus and Ficus palmata is described, the poly (D,L-lactide-co-glycolide), curcumin and organic solvent acetone used in step (a) is 100 mg, 5 mg and 10 ml, respectively.

In yet another aspect of the present invention, 8 formulations of poly (D,L-lactide-co-glycolide) nanoparticles have been characterized by ultraviolet visible spectroscopy, transmission electron microscopy, dynamic light scattering and high performance liquid chromatography. Detailed information about these formulations are listed in Table 1 and 2.

In still another aspect, present invention used four curcumin loaded poly (D,L-lactide-co-glycolide) for studying in vitro release profile of curcumin.

In yet another aspect of the present invention, increased photo-stability of curcumin loaded Camellia sinensis leaf extracts synthesized loaded poly (D,L-lactide-co-glycolide) nanoparticles was checked by high performance liquid chromatography.

In still another aspect of the present invention, increase in curcumin solubility of curcumin loaded Camellia sinensis leaf extracts synthesized loaded poly (D,L-lactide-co-glycolide) nanoparticles was measured by high performance liquid chromatography.

In yet another aspect of the present invention, of curcumin loaded Camellia sinensis leaf extracts synthesized loaded poly (D,L-lactide-co-glycolide) nanoparticles showed approximately similar anti-oxidant activity at half curcumin concentration (Table 3)

TABLE 1
Detailed information about the synthesis of blank
PLGA and CURCU loaded PLGA NPs.

| | Plant | | | | | | |
|---|---|---|---|---|---|---|---|
| | leaf | | | | | | |
| | extract | | | | DLS | Zeta | TEM |
| Sample | (LE) | Acetone | CURCU | PLGA | size | potential | Size |
| Code | (ml) | (ml) | (mg) | (mg) | (nm) | (mV) | (nm) |
| CSE- | 20 | 10 | — | 100 | 110 ± 5 | −0.32 ± 0.3 | 68 ± 12 |
| PLGA | | | | | | | |
| BE- | 20 | 10 | — | 100 | 220.4 ± 15 | 0.14 ± 0.4 | 126 ± 21 |
| PLGA | | | | | | | |
| RE- | 20 | 10 | — | 100 | 127 ± 10 | −0.12 ± 0.5 | 132 ± 26 |
| PLGA | | | | | | | |
| FE-PLGA | 20 | 10 | — | 100 | 215 ± 17 | 0.17 ± 0.029 | 122 ± 11 |
| CSE- | 20 | 10 | 5 | 100 | 161 ± 10 | −0.34 ± 0.3 | 90 ± 10 |
| CURCU- | | | | | | | |
| PLGA | | | | | | | |
| BE- | 20 | 10 | 5 | 100 | 476.2 ± 20 | −36.3 ± 2 | 116 ± 21 |
| CURCU- | | | | | | | |
| PLGA | | | | | | | |
| RE- | 20 | 10 | 5 | 100 | 145 ± 5 | −0.36 ± 0.03 | 206 ± 20 |
| CURCU- | | | | | | | |
| PLGA | | | | | | | |
| FE- | 20 | 10 | 5 | 100 | 327 ± 25 | −0.34 ± 0.03 | 158 ± 42 |
| CURCU- | | | | | | | |
| PLGA | | | | | | | |

TABLE 2
Encapsulation efficiency and drug loading of CSE-CURCU-PLGA, BE-
CURCU-PLGA, RE-CURCU-PLGA and FE-CURCU-PLGA NPs
calculated on the basis of HPLC calibration curve.

| Formulation | EE (%) | Drug loading (%) |
|---|---|---|
| CSE-CURCU-PLGA | 92.6 ± 3 | 9.8 |
| BE-CURCU-PLGA | 98.9 ± 2 | 8.5 |
| RE-CURCU-PLGA | 97.6 ± 2 | 15.6 |
| FE-CURCU-PLGA | 95.4 ± 2 | 7.8 |

TABLE 3
DPPH assay of pure CURCU and CSE-CURCU-PLGA NPs

| Conc. (mg/ml) | OD (517 nm) | Scavenging activity |
|---|---|---|
| Pure CURCU (0.25) | 0.082 | 88.5 |
| CSE-CURCU-PLGA | 0.084 | 88.3 |
| (0.049) | | |

### EXAMPLES

The following examples, which include preferred embodiments, will serve to illustrate the practice of this invention, it being understood that the particulars shown are by way of examples and for purpose of illustrative discussion of preferred embodiments of the invention only and are not limiting the scope of the invention.

### Example 1

Preparation of Camellia sinensis Leaf Extracts

The Camellia sinensis leaves were collected from CSIR-IHBT, Palampur campus Bharmaat Palampur, Pin—176061, Kangra Himachal Pradesh, located at geographical coordinates 32° 06′18″ N and 76° 33′22″ E and at a height of about 1260 meters above mean sea level. Forty gram freshly crushed leaves of Camellia sinensis were dissolved in 500 ml double distilled water (DDW) and boiled for ˜30 minutes. The above solution was cooled to room temperature (25-30° C.) and filtered through Whatman filter paper and termed as Camellia sinensis leaf extract (CSE). The supernatant was used as such for the synthesis of PLGA NPs.

### Example 2

Preparation of Dendrocalamus hamiltonii Leaf Extracts

The Dendrocalamus hamiltonii leaves were collected from CSIR-IHBT, Palampur campus Bharmaat Palampur, Pin—176061, Kangra Himachal Pradesh, located at geographical coordinates 32° 06′18″ N and 76° 33′22″ E and at a height of about 1260 meters above mean sea level. Forty gram freshly crushed leaves of Dendrocalamus hamiltonii were dissolved in 500 ml double distilled water (DDW) and boiled for ˜30 minutes. The above solution was cooled to room temperature (25-30° C.) and filtered through Whatman filter paper and termed as Dendrocalamus hamiltonii leaf extract (BE). The supernatant was used as such for the synthesis of PLGA NPs.

### Example 3

Preparation of Rubus ellipticus Leaf Extracts

The Rubus ellipticus leaves were collected from CSIR-IHBT, Palampur campus Bharmaat Palampur, Pin—176061, Kangra Himachal Pradesh, located at geographical coordinates 32° 06′18″ N and 76° 33′22″ E and at a height of about 1260 meters above mean sea level. Forty gram freshly crushed leaves of Rubus ellipticus were dissolved in 500 ml double distilled water (DDW) and boiled for ˜30 minutes. The above solution was cooled to room temperature (25-30° C.) and filtered through Whatman filter paper and termed as Rubus ellipticus leaf extract (RE). The supernatant was used as such for the synthesis of PLGA NPs.

### Example 4

Preparation of Ficus palmata Leaf Extracts

The Ficus palmata leaves were collected from CSIR-IHBT, Palampur campus Bharmaat Palampur, Pin—176061, Kangra Himachal Pradesh, located at geographical coordinates 32° 06′18″ N and 76° 33′22″ E and at a height of about 1260 meters above mean sea level. Forty gram freshly crushed leaves of Ficus palmata were dissolved in 500 ml double distilled water (DDW) and boiled for ˜30 minutes. The above solution was cooled to room temperature (25-30° C.) and filtered through Whatman filter paper and termed as Ficus palmata leaf extract (FE). The supernatant was used as such for the synthesis of PLGA NPs.

### Example 5

### Synthesis of Blank PLGA Nanoparticles Using CSEs

Briefly PLGA (100 mg) was dissolved in 10 ml of acetone and stirred for 2 hours. Twenty ml of CSE was added to the above solution at 5:1 (w/v) and the solution was kept under stirring at 40-50° C. for 6-10 hours. The acetone was removed using rotavapor. The solution was centrifuged at 13500 rpm at 10° C. for 20 minutes for collecting CSE-PLGA NPs.

### Example 6

### Synthesis of Curcumin-PLGA Nanoparticles Using CSEs

Briefly PLGA (100 mg) and 5 mg curcumin was dissolved in 10 ml of acetone and stirred for 2 hours. Twenty ml of CSE was added to the above solution containing PLGA and curcumin and the solution was kept under stirring at 40-50° C. for 6-10 hours. The acetone was removed using rotavapor. The solution was centrifuged at 13500 rpm at 10° C. for 20 minutes for collecting CSE-CURCU-PLGA NPs.

### Example 7

### Synthesis of Blank PLGA Nanoparticles Using BEs

Briefly PLGA (100 mg) was dissolved in 10 ml of acetone and stirred for 2 hours. Twenty ml of BE was added to the above solution at 5:1 (w/v) and the solution was kept under stirring at 40-50° C. for 6-10 hours. The acetone was removed using rotavapor. The solution was centrifuged at 13500 rpm at 10° C. for 20 minutes for collecting BE-PLGA NPs with a ratio of polymer to leaf extracts is in the range of 5:1 (w/v).

### Example 8

### Synthesis of Curcumin-PLGA Nanoparticles Using BEs

Briefly PLGA (100 mg) and 5 mg curcumin was dissolved in 10 ml of acetone and stirred for 2 hours. Twenty ml of BE was added to the above solution containing PLGA and curcumin and the solution was kept under stirring at 40-50° C. for 6-10 hours. The acetone was removed using rotavapor. The solution was centrifuged at 13500 rpm at 10° C. for 20 minutes for collecting BE-CURCU-PLGA NPs.

### Example 9

### Synthesis of Blank PLGA Nanoparticles Using REs

Briefly PLGA (100 mg) was dissolved in 10 ml of acetone and stirred for 2 hours. Twenty ml of RE was added to the above solution at 5:1 (w/v) and the solution was kept under stirring at 40-50° C. for 6-10 hours. The acetone was removed using rotavapor. The solution was centrifuged at 13500 rpm at 10° C. for 20 minutes for collecting RE-PLGA NPs with a ratio of polymer to leaf extracts is in the range of 5:1 (w/v).

### Example 10

### Synthesis of Curcumin-PLGA Nanoparticles Using REs

Briefly PLGA (100 mg) and 5 mg curcumin was dissolved in 10 ml of acetone and stirred for 2 hours. Twenty ml of RE was added to the above solution containing PLGA and curcumin and the solution was kept under stirring at 40-50° C. for 6-10 hours. The acetone was removed using rotavapor. The solution was centrifuged at 13500 rpm at 10° C. for 20 minutes for collecting RE-CURCU-PLGA NPs.

### Example 11

### Synthesis of Blank PLGA Nanoparticles Using FEs

Briefly PLGA (100 mg) was dissolved in 10 ml of acetone and stirred for 2 hours. Twenty ml of FE was added to the above solution at 5:1 (w/v) and the solution was kept under stirring at 40-50° C. for 6-10 hours. The acetone was removed using rotavapor. The solution was centrifuged at 13500 rpm at 10° C. for 20 minutes for collecting FE-PLGA NPs with a ratio of polymer to leaf extracts is in the range of 5:1 (w/v).

### Example 12

### Synthesis of Curcumin-PLGA Nanoparticles Using FEs

Briefly PLGA (100 mg) and 5 mg curcumin was dissolved in 10 ml of acetone and stirred for 2 hours. Twenty ml of FE was added to the above solution containing PLGA and curcumin and the solution was kept under stirring at 40-50° C. for 6-10 hours. The acetone was removed using rotavapor. The solution was centrifuged at 13500 rpm at 10° C. for 20 minutes for collecting FE-CURCU-PLGA NPs.

### Example 13

### Encapsulation Efficiency of CURCU Loaded PLGA Nanoparticles Synthesized Using CSEs, BEs, REs and FEs.

Encapsulation efficiency of CURCU in CSE-CURCU-PLGA, BE-CURCU-PLGA, RE-CURCU-PLGA and FE-CURCU-PLGA was measured using validated HPLC method. The supernatant solution of all the above NPs was filtered through 0.22 μm filter. The solution was directly injected to HPLC. The reverse phase C18 column (150 mm×4.6 mm, 5 μm pore size) was used for HPLC separation. Acetonitrile and water (48:52) were used as mobile phase with flow rate of 1 ml/min. The detection wavelength was selected as 420 nm. The calibration curve was drawn by preparing different amount of CURCU (0.023-0.75 mg/mL) vs. peak area of eluted peak.

### Example 14

### Characterization of CURCU-Loaded PLGA NPs

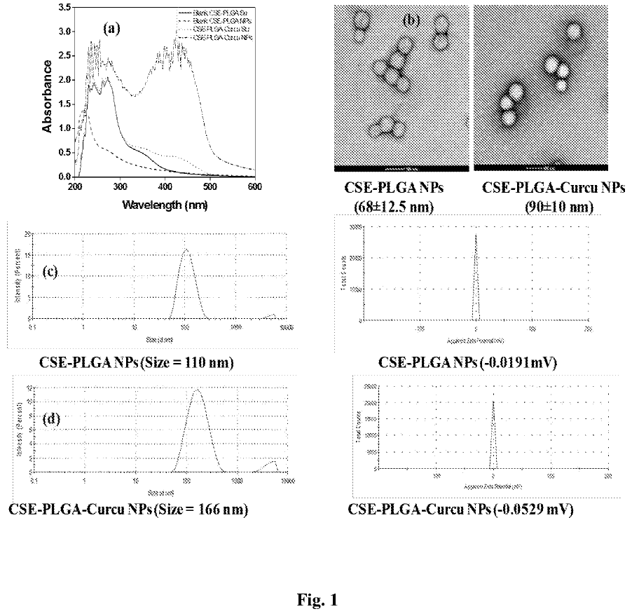

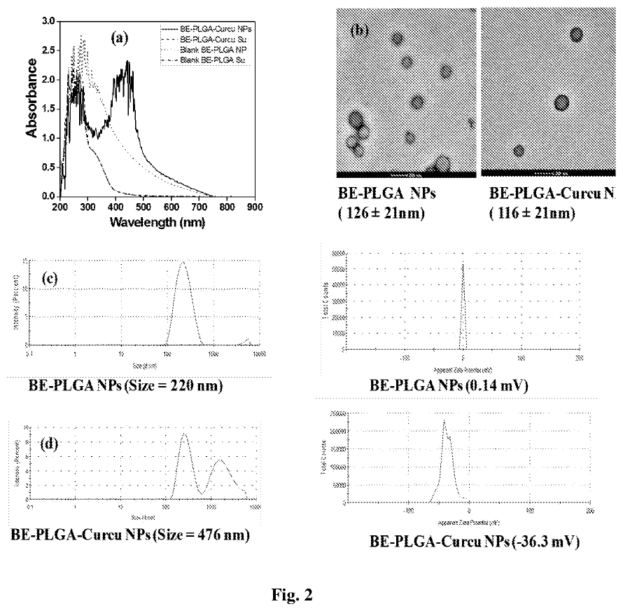

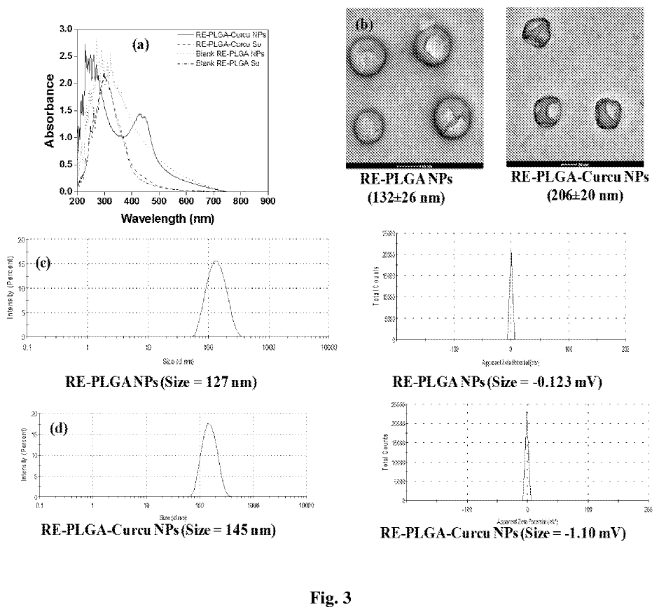

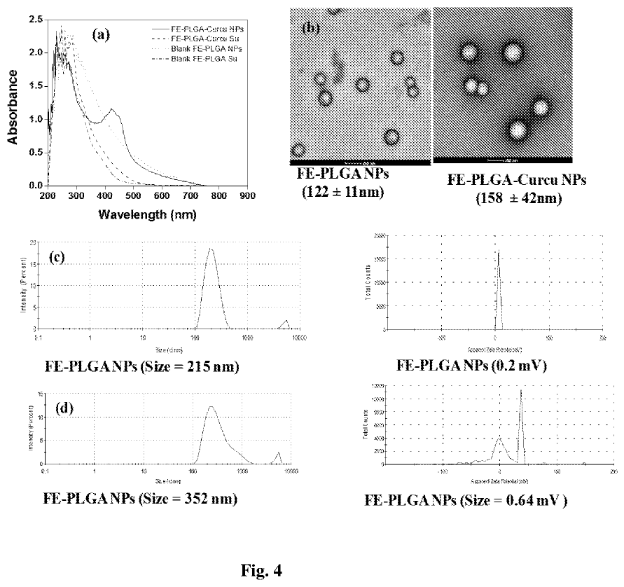

CSE-PLGA, CSE-CURCU-PLGA, BE-PLGA, BE-CURCU-PLGA, RE-PLGA, RE-CURCU-PLGA, FE-PLGA and FE-CURCU-PLGA were characterized by UV-Vis, TEM and DLS (Table 1, FIG. 1-4).

### Example 15

In Vitro Release Profile of CURCU from CSE-CURCU-PLGA NPs

In vitro release studies of CURCU from CSE-CURCU-PLGA NPs was performed by incubating 3.4 mg of CSE-CURCU-PLGA NPs in 15 ml of 0.1 M PBS at pH 7.4. At pre-selected times (0, 0.5, 1, 2, 4, 6, 8, 12, 24, 48, 72, 96, 120, 144 and 168 h), 1.0 mL of sample was taken and lyophilized. This lyophilized solution was dissolved in 100% acetonitrile. The released CURCU was quantified with the help of validated HPLC method.

### Example 16

In Vitro Release Profile of CURCU from BE-CURCU-PLGA NPs

In vitro release studies of CURCU from BE-CURCU-PLGA NPs was performed by incubating 2.5 mg of BE-CURCU-PLGA NPs in 15 mL of 0.1 M PBS at pH 7.4. At pre-selected times (0, 0.5, 1, 2, 4, 6, 8, 12, 24, and 48 h), 1.0 mL of sample was taken and lyophilized. This lyophilized solution was dissolved in 100% acetonitrile. The released CURCU was quantified with the help of validated HPLC

### Example 17

In Vitro Release Profile of CURCU from RE-CURCU-PLGA NPs

In vitro release studies of CURCU from RE-CURCU-PLGA NPs was performed by incubating 2.2 mg of RE-CURCU-PLGA NPs in 15 mL of 0.1 M PBS at pH 7.4. At pre-selected times (0, 0.5, 1, 2, 4, 6, 8, 12, 24, and 48 h), 1.0 mL of sample was taken and lyophilized. This lyophilized solution was dissolved in 100% acetonitrile. The released CURCU was quantified with the help of validated HPLC.

### Example 18

In Vitro Release Profile of CURCU from FE-CURCU-PLGA NPs

In vitro release studies of CURCU from FE-CURCU-PLGA NPs was performed by incubating 2.2 mg of FE-CURCU-PLGA NPs in 15 mL of 0.1 M PBS at pH 7.4. At pre-selected times (0, 0.5, 1, 2, 4, 6, 8, 12, 24, and 48 h), 1.0 mL of sample was taken and lyophilized. This lyophilized solution was dissolved in 100% acetonitrile. The released CURCU was quantified with the help of validated HPLC.

### Example 19

### Photo-Stability Studies of Pure CURCU and CSE-CURCU-PLGA

Photo-stability studies were done by incubating 0.03 mg of pure CURCU and 2 mg of CSE-CURCU-PLGA NPs in 1 mL of acetonitrile. The resulting solution was irradiated under laser (633 nm) for half an hour. The % degradation of CURCU was analyzed using HPLC method.

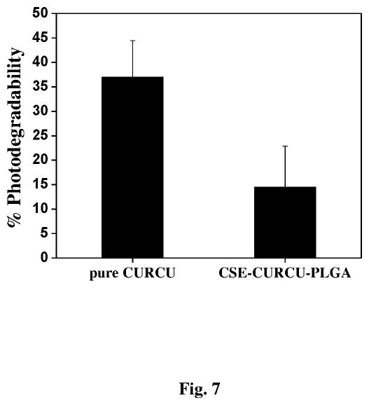

The study showed 22±7.5% increased photo-stability of CURCU in CSE-CURCU-PLGA over pure CURCU (FIG. 7).

### Example 20

### Solubility Studies of Pure CURCU and CSE-CURCU-PLGA in Double Distilled Water

Solubility study was performed by incubating 2 mg of pure CURCU and CSE-CURCU-PLGA in 2 ml of double distilled water. Amount of dissolved CURCU in water was analyzed using HPLC method.

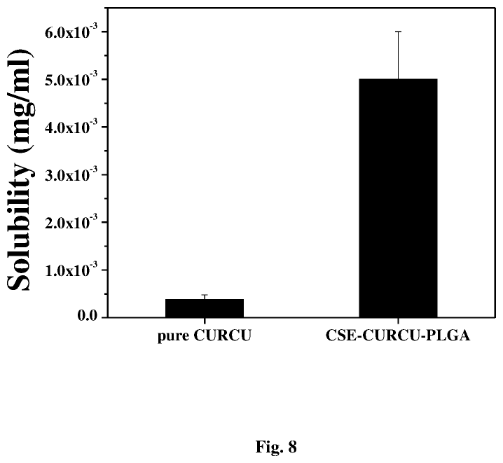

The study showed 13±5 folds increased solubility of CURCU in CSE-CURCU-PLGA over pure CURCU (FIG. 8).

### Advantages

(1) The use of leaf extracts of plants viz., Camellia sinensis, Dendrocalamus hamiltonii, Rubus ellipticus and Ficus palmata will reduce the toxicity concerns associated with the PLGA nanoparticles.
(2) The use of leaf extracts will reduce the cost of synthesis of PLGA NPs by nanoprecipitation method.
(3) The plants viz., Camellia sinensis, Dendrocalamus hamiltonii, Rubus ellipticus and Ficus palmata have medicinal importance which will enhance the therapeutic importance of the synthesized PLGA NPs.
(4) Plant leaf synthesized PLGA NPs are stable.
(5) The described process can be used for the encapsulation of many other medicinally important molecules.
(6) Plant leaf extract synthesized PLGA NPs can be used for slow and sustained release of other antioxidant molecules.
(7) Aqueous solubility and photo-stability of curcumin was increased.

1. Stabilized polymeric nanoparticles, having particle size in the range of 68 nm to 206 nm, synthesized using leaf extracts as stabilizers, wherein a ratio of polymer to leaf extracts in the stabilized polymeric nanoparticles is in the range of 5:1 (w/v).

2. The stabilized polymeric nanoparticles of claim 1, wherein the leaf extract is obtained from plants selected horn the group consisting of Camellia sinensis, Dendrocalamus hamiltonii, Ficus palmata and Rubus ellipticus.

3. The stabilized polymeric nanoparticles of claim 1, wherein the polymer is poly (D,L-lactide-co-glycolide) (PLGA).

4. The stabilized polymeric nanoparticles of claim 1, wherein polymeric nanoparticles are loaded with 7.8% to 15.6% curcumin.

5. The stabilized polymeric nanoparticles of claim 4, having increased solubility of 13±5 folds and photo-stability of 22±7.5%.

6. The stabilized polymeric nanoparticles of claim 4, having slow and sustained release of curcumin of up to 45% after 4 hours.

7. A process for the synthesis of stabilized PLGA nanoparticles according to claim 3, the process comprising:
(a) dissolving freshly crushed leaves in double distilled water (DDW) and boiling up to 30 minutes;
(b) cooling the solution to room temperature (25-30° C.) and filtering to obtain a supernatant comprising plant extract;
(c) dissolving poly (D,L-lactide-co-glycolide) PLGA in acetone under stirring for 2 hours to obtain a PLGA solution;
(d) adding the supernatant obtained in (b) to the PLGA solution of (c) and stirring at a temperature from 40° C. to 50° C. for 6 hours to 10 hours, wherein PLGA:plant extract is at a ratio of 5:1 (w/v); and
(e) removing acetone and centrifuging at about 13500 rpm at 10° C. for 20 minutes to collect stabilized PLGA nanoparticles.

8. The process according to claim 7, wherein the leaves are obtained from plants selected from the group consisting of Camellia sinensis, Dendrocalamus hamiltonii, Ficus palmate, and Rubus ellipticus.

9. The process according to claim 7, further comprising loading curcumin in PLGA nanoparticles by dissolving curcumin along with the PLGA in acetone at a ratio of PLGA to curcumin at 100:5.

10. Curcumin loaded stabilized PLGA nanoparticles synthesized by the process according to claim 9.
